(19) United States
(12) Patent Application Publication
(10) Pub. No.: US 2010/0248228 A1
(43) Pub. Date: Sep. 30, 2010

(54) Method for detecting DNA methylation in cancer cells

(75) Inventors: Michael Xia Wang, Columbia, MO (US); Huidong Shi, Martinez, GA (US); Srilatha Nalluri, Augusta, GA (US)

Correspondence Address: UNIVERSITY OF MISSOURI SYSTEM;OFFICE OF INTELLECTUAL PROPERTY ADMIN., 475 MCREYNOLDS HALL, COLUMBIA, MO, 65211-2015, US

(73) Assignee: The Curators of the University of Missouri, Columbia, MO (US)

(21) Appl. No.: 12/383,790

(22) Filed: Mar. 27, 2009

Related U.S. Application Data
(60) Provisional application No. 61/072,064, filed on Mar. 27, 2008.

(51) Int. Cl. C12Q 1/68

(52) U.S. Cl. 435/6

(57) ABSTRACT

The present invention provides a detecting method of detecting malignant cells in a patient's specimen or a biological sample. Specifically, the inventive method includes the steps of extracting a genomic DNA, digesting said genomic DNA with one or multiple methylation sensitive restriction enzymes, and amplifying by PCR with one or multiple selected primers. The PCR can be performed in a conventional or a real-time platform. The inventive method can detect leukemia cells in 90% ALL patients at a sensitivity of up to 10⁻⁶. The inventive method also provides broad clinical applications in cancer (including hematopoietic and solid tumors) screening and risk assessment, early detection and diagnosis confirmation, and therapeutic monitoring, minimal residual disease detection and prognostic prediction.

### CROSS-REFERENCE TO RELATED APPLICATIONS

This application claims the benefit of U.S. Provisional Application Ser. No. 61/072,064, entitled “Method for Detecting DNA Methylation in Cancer Cells,” to Wang, et al., filed on Mar. 27, 2008.

### BACKGROUND OF INVENTION

1. Field of the Invention

The present invention relates to a cancer detection method based upon DNA methylation differences at specific CpG sites. More specifically, the present invention relates to a diagnostic method for detecting cancer cells in cancer patients, especially for acute lymphoblastic leukemia.

2. Background Art

Methods in Detecting Aberrant DNA Methylation

In the past decade, the studies in the cancer epigenetic area have identified hundreds of aberrant DNA methylation loci in virtually all types of cancers including hematopoietc tumors. DNA hypermethylation usually occurs in CG rich promoter region and/or exon 1 region (CpG island) of a gene and is tumor specific and inheritable. If the hypermethylation occurs in a tumor suppressor gene or other tumorigenesis relevant genes, the gene will be permanently silenced. Among these genes, DLC-1, PCDHGA 12 and CDH1 have garnered many interests in cancer research. In 2002, DLC-1 promoter hypermethylation was reported in solid tumor cell lines such as liver, colon, and prostate cancers; In 2005, the PCDHGA 11 gene methylation (similar to that of PCDHGA 12) was reported in brain tumor astrocytoma, and CDH1 (E-cadherin) gene methylation has been reported in almost all types of tumors. The inventors' lab also reported DLC-1 and PCDHGA 12 gene methylation patterns in various hematopoietic tumors including acute lymphoblastic leukemia (ALL).

Currently there is a verity of methods and studies in attempting to identify hypermethylation sites in cancer cells. For example, a technique disclosed in the U.S. Pat. No. 7,037,650 B2 granted to Gonzalgo that provides for a bisulfite treatment of a genomic DNA, followed by PCR (MSP) or methylation-sensitive single nucleotide primer extension (Ms-SnuPE), for determination of strand-specific methylation status at cytosine residues. However, bisulfite destroys the majority of DNA during the treatment.

A study done by Singer et al in 1990 also reported a methylation-sensitive restriction enzyme HpaII-based method to assay DNA methylation. In this approach, a genomic DNA sample is treated with a single enzyme, Hpa II, using a complicated guanidine HCL procedure, followed by PCR amplification. However, this approach has not been widely adopted at clinical setting because of a high degree of false positive signal resulted by incomplete enzyme digestion.

Therefore, there is a need to provide a novel and improvement method in determining DNA methylation patterns with simple procedure, high accuracy, and suitable for clinical usage.

Detection of Minimal Residue Disease

Current diagnosis of cancer is largely based on capability to identify biological cancer cells in the patient biopsy specimens. In the case of acute lymphoblastic leukemia (ALL), by combination of morphological evaluation, flow cytometric immunophenotyping, and molecular clonality analysis in blood and/or bone marrow specimens, most cases can be diagnosed correctly. After induction chemotherapy, however, to determine if the patients are in true remission is problematic. Leukemic blasts are not always distinguishable from normal hematopoietic blasts in a recovery marrow microscopically. Even with the best knowledge of morphological evaluation, the complete remission is defined as that the leukemia blasts are less than 5% of nuclear bone marrow cells. With this definition, a given patient could harbor up to 10¹⁰ leukemia cells after initial induction. If these residual leukemic cells are left with no further treatment, most patients will relapse.

The presence of submicroscopic leukemia cells that can be detected using more sensitive methods is defined as minimal residual disease (MRD). Detection of MRD during entire disease course in ALL patients has several clinical utilities. First, it is an objective parameter to directly measure the early respond to chemotherapy; secondly, the status of MRD is then used to stratify the risk groups of the patients in most modern treatment protocols; thirdly, it is a confirmed prognostic factor prior to hematopoietic stem cell transplantation (HSCT); and finally, it is the best predictive factor for the relapse. The level of MRD to reliably predict relapse has been reported as 10⁻³ or 10⁻⁴ in recent large clinical studies.

Current MRD detection in clinical laboratories of the large medical centers or reference laboratories is mainly using three methodologies: multiparameter flow cytometric immunophenotyping, real-time quantitative PCR (RQ-PCR)-based detection of fusion gene transcripts and RQ-PCR-based detection of clonal immunoglobulin gene (Ig) and T-cell receptor (TCR) gene rearrangements. Immunophenotypic analysis uses multicolor flow-cytometry to detect aberrant or leukemia-associated antigens that are not expressed on normal hematopoietic or lymphoid progenitors. RQ-PCR-based detection of leukemia-associated fusion genes relies on the presence of specific chromosomal translocations in subset of ALL. RQ-PCR-based detection of antigen receptor gene rearrangements is based on the presence of clonal fingerprint-like sequences in junctional region of antigen receptor genes such as (V-(D)-J) of Ig (H and K) and TCR (γ and δ) genes in vast majority of the ALL patients. Although these methods have high sensitivity (10⁻⁴ to 10⁻⁵) and specificity, they are unable to identify the residual leukemic cells that lack initial specific detectible chromosomal translocations or with ongoing somatic mutations resulting in clonal evolution or antigen shifting. In addition, the technical complexity, poor reproducibility at higher sensitivity, and high cost are the major obstacles for the routine clinical application.

Therefore, there is a need for a new diagnostic method for detecting MRD, such as, the residual leukemia cells in ALL patient specimens, with speedy testing procedure and improved sensitivity.

### SUMMARY OF INVENTION

In one aspect of the invention, a novel diagnostic method for detecting malignant cells in a cancer patient, especially MRD in patient specimens using the specific DNA methylations as biomarkers, is described. According to one embodiment of the invention, the inventive method for determining DNA methylation status at cytosine sites comprises the steps of 1) obtaining genomic DNA from a biological sample to be assayed, 2) treating said genomic DNA with at least one pre-selected methylation sensitive restriction enzyme to result a sample of digested DNA, 3) performing a PCR amplification procedure on said digested DNA with at least one pre-selected primer set, and 4) determining said DNA methylation status by comparing with a pair of preselected positive and negative controls.

According to one embodiment of the inventive method, the DNA sample may be collected from a patient's blood, bone marrow, tissue, or other specimens; the methylation sensitive enzyme may be selected from Aci I, Hap II, HinP1 I, BstU I, Hha I, Tai I, or any combination thereof; and the primers may be selected from DLC-1 primers, CDH1 primers, PCDHGA12 primers, or any combination thereof. The preferred internal control for PCR amplification is β-actin primers. The positive control may be any known tumor cell line DNA or Sss I methyltransferase-treated normal human DNA. The negative control may be any normal human blood or bone marrow cell DNA.

According to another embodiment of the invention, the aforesaid performing a PCR amplification procedure may be included a step of performing a PCR procedure on said digested DNA with a sequential set of pre-selected primers. According to one embodiment, the performing step includes the sub steps of amplifying said digested DNA with a first pre-selected primer to result a first PCR product with long fragment, and then amplifying said first PCR product with a second pre-selected primer to result a second or nested PCR product with short fragment.

According to yet another embodiment of the invention, the aforesaid performing a PCR amplification step may include a step of performing a multiplex PCR on said digested DNA with the combinations of two or more pre-selected primers.

According to still yet another embodiment of the invention, the aforesaid performing a PCR amplification step may further include the steps of carrying out a real-time PCR procedure to quantitatively determine percentage of the malignant cells in a testing sample.

In another aspect of the invention, the inventive method may be employed as a method or kit in various clinical applications, such as cancer screening and risk assessment, early detection and diagnosis confirmation, therapeutic monitoring and prognostic prediction, and minimal residual diseases detection.

### BRIEF DESCRIPTION OF DRAWINGS

FIG. 1 is a flow chat illustrating the inventive method, according to one embodiment of the invention.

FIG. 2 illustrates two sets of tests to evaluate DNA digestion efficiency by different methylation sensitive enzymes and their combinations.

FIG. 3A is a schematic of the DLC-1 promoter CpG island region of interest in leukemia cells; FIG. 3B is PCR amplifications illustrating their methylation map in leukemia cells employing the inventive method and the methylation densities in three regions; FIG. 3C is the PCR amplifications of hypermethylation in colon cancer cell line employing the inventive method.

FIG. 4A illustrates three PCR amplifications of hypermethylation of 21 B-ALL patients' bone marrow specimens each with a preselected primer targeting different genes; FIG. 4B illustrates the multiplex PCR amplification of the same samples with all three primers.

FIGS. 5A-C illustrates the analytic sensitivity of the inventive method.

FIG. 6 illustrates a real-time PCR with the inventive method.

FIG. 7 illustrates detection of hypermethylation in selected leukemia cell lines using the inventive method.

FIG. 8 illustrates detection of DLC-1 methylation patterns in 26 B-ALL patients using the inventive method.

### DETAILED DESCRIPTION OF INVENTION

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

The present invention provides a method for determining DNA methylation patterns at a cytosine residue of a CpG sequence by enzyme digestion of a sample of genomic DNA with one or multiple pre-selected methylation sensitive restriction enzymes followed by PCR amplification with one or multiple pre-selected primers. The invention teaches that there is fundamental difference between malignant and normal cells in their methylomes. When a genomic DNA sample with malignant and normal cells is subjected for methylation sensitive enzyme restriction, their patterns of digestion will be different. Specific hypermethylation regions in malignant cells are resistant to digestion and remain as large intact fragments; while same regions in normal cells are digested to small fragments. The specific hypermethylation loci in malignant cells can be differentially detected by various PCR amplifications. In other words, these loci can be used as the biomarkers to detect malignant cells in patient specimens. The invention also stresses that the complete digestion, which can be sufficiently achieved employing a combination of several selected methylation sensitive enzymes, is critical to avoid false positive and to ensure an accurate detection.

In a specific aspect, the inventive method comprise the steps of 1) obtaining genomic DNA from a biological sample, 2) treating said genomic DNA with at least one pre-selected methylation sensitive enzyme to result a sample of digested DNA, 3) performing a PCR amplification procedure on said digested DNA with at least one set pre-selected primers to result a PCR product, and 4) determining said DNA methylation status by comparing said PCR product with a pair of preselected positive and negative controls.

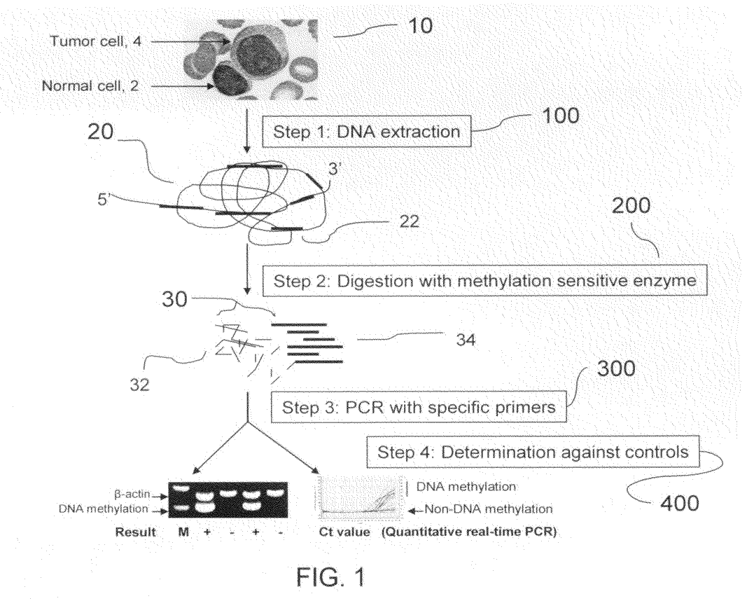

Refer to FIG. 1, which is a flow chat illustrating the inventive method. In FIG. 1, a biological sample, 10, is shown having normal cells, 2, and tumor (malignant) cells, 4. Step 1, 100, is the DNA extraction with any standard method currently available, which produces a genomic DNA sample, 20, with aberrant hypermethylation regions, 22. Step 2, 200, is the DNA digestion with preselected methylation sensitive enzyme(s) to produce digested DNA fragments, 30, with small fragments, 32, from the normal cells and larger intact fragments, 34, from the tumor cells. Step 3, 300, the PCR amplification with specific primers on the digested DNA fragments, 30, where only the larger intact fragments, 34, will be amplified to product PCR products. Step 4, 400, the determination of methylation status by comparing the PCR products with preselected controls in either gel electrophoresis or real-time fluorescence signal format.

In the aforesaid obtaining genomic DNA step, a genomic DNA sample may be extracted from variety of patient specimens, such as blood sample, bone marrow sample, or tissue sample, or any other biological samples. The extraction process may adopt any standard DNA extraction protocol currently available in the medical and biological fields. For example, a genomic DNA may be extracted from a blood sample with QIAamp DNA Blood mini kit (Qiagen, Valencia, Calif.) according to the manufacture instruction.

In the aforesaid treating genomic DNA step, the methylation sensitive enzymes may be any known methylation sensitive enzymes, for example: Aci I, Hap II, HinP1 I, BstU I. Hha I, Tai I, or any combination thereof. According to one embodiment of the invention, to ensure a complete digestion, preferably the genomic DNA sample can be treated first with a set of three methylation sensitive enzymes Aci I, Hap II, and HinP1 I at a standard temperature around 37° C., and then with another enzyme, BstU I, at a elevated temperature around 60° C.

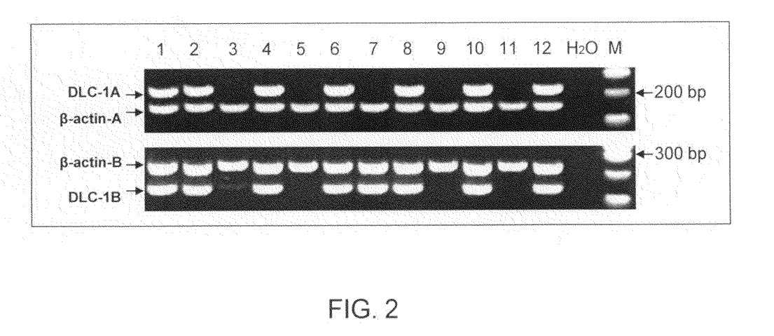

To ensure a complete digestion, the invention selects the target regions which contain multiple restriction sites for multiple methylation sensitive enzymes. For example, within the target region A of DLC-1 CpG island using DLC-1A primer set, there are eight restriction sites including three BstU I, two Aci I, two Hinp1 I and one Hpa II restriction sites, respectively. The invention has examined the digestion efficiency using a single selected methylation enzyme and a combination of four selected enzymes compared with same amount of normal human blood cell (250 ng, odd number lanes) and NALM-6 leukemia cell line DNA (250 ng, even number lanes), and the results are shown in FIG. 2. Effectiveness of digestion was determined using DLC-1A primer set (upper panel gel) or DLC-1B primer set (lower panel gel) at a standard condition. Methylation sensitive enzymes Hpa II (lane 5) and BstU I (lane 9) gave a complete digestion, respectively; Aci I (lane 3) showed a partial digestion (50% digestion rate in NEBuffer 4), and Hinp1I (lane 7) and controls (lane 1 and 2, no enzyme) showed no digestion in lower panel gel since there is no restriction site for Hinp1I in the target region B. The combined enzymes demonstrated an absolutely complete digestion (lane 11). PCR reaction internal control β-actin band (257 bp) was seen in each lane, but not in the water negative control (lane 13). Thus, the invention prefers to employ a set of four above selected enzymes to ensure a complete digestion.

In the aforesaid performing PCR amplification with primer step, the primers are designed to target one or multiple specific hypermethylated regions. In a specific aspect, the invention selected primers to target hypermethylation on the DLC-1, CDH1, or PCDHGA12 genes. According to one embodiment of the invention, several sets of primers are designed to target the CpG island region of DLC-1 gene, since the DLC-1 gene is a tumor suppressor gene that has been reported having CpG island methylation in many hematopoietic malignancies and solid tumors.

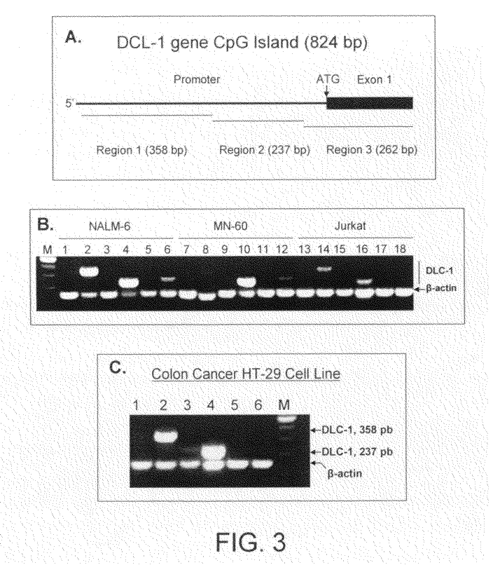

Refer to FIG. 3A, which illustrates the schematic of the DLC-1 promoter CpG island region of interest in human genome. The DLC-1 CpG island expands 824 by from the 5′untranslational region to the first part of exon 1 of a DLC-1 gene. The DLC-1 CpG island is artificially separated into three regions: Region 1 with 358 bp from the 5′-end, Region 2 with 237 by in the middle, and Region 3 with 262 by ending at Exon 1. Three sets of primers to cover the entire DLC-1 CpG island are:

Primer set 1: SEQ ID Nos. 1 and 2, targeting Region 1;

Primer set 2: SEQ ID Nos. 3 and 4, targeting Region 2; and

Primer set 3: SEQ ID Nos. 5 and 6, targeting Region 3.

The invention further finds that the Region 2 of the DLC-1 CpG island is the region with relatively high methylation density. Refer to FIG. 3B, which illustrates the methylation patterns at the three regions on the DLC-1 gene in three ALL cell lines. The three cell lines are NALM-6 (lanes 2, 4, 6), MN-60 (lanes 8, 10, 12), and Jurkat (lanes 14, 16, 18), while the odd lanes (1, 3, 5, 7, 9, 11, 13, 15, 17) are the negative controls (normal human blood cell DNA). The PCR amplification was performed with a combination of the above listed three primer sets. Lanes 2, 8, and 14 represent the methylation density of the Region 1 in all three cell lines, Lanes 4, 10, and 16 the Region 2; and Lanes 6, 12, and 18 Region 3. Based on the FIG. 3B, the Region 2 of the DLC-1 gene provides relatively high methylation density. Thus, as an alternatively of using a combination of primer sets, the Primer set 2 (SEQ ID Nos. 3 and 4) or Primer set 8 (SEQ ID Nos 15 and 16) targeting the Region 2 may be used alone to sufficiently and accurately detect the presence of malignant cells, which may provide a simply and speedy clinical testing procedure.

The finding that the relatively high methylation density in the Region 2 on the DLC-1 gene is confirmed by the similar testing performed with the colon cancer cell line HT-29, as shown in FIG. 3C. In FIG. 3C, odd lanes (1, 3, 5) are the negative controls; Lane 2 represents the methylation density of Region 1; Lane 4 Region 2; and Lane 6 Region 3. FIG. 3C also shows that the signal at Region 2 (Lane 4) is the relatively highest among the regions in colon cancer cells.

Similarly, the specific primers may be designed to target DNA methylation in PCDHGA 12A and CDH1 gene CpG islands. For example, the primer sets (SEQ ID Nos 7 and 8) target PCDHGA 12A, while another primer sets (SEQ ID Nos. 9 and 10) target CDH1. The digested DNA may be amplified with one or any combinations of the three selected primer sets targeting all three genes (such as a multiplex PCR described later). For the internal control, the 13-actin primer sets (SEQ ID Nos. 11 and 12) may also be co-amplified.

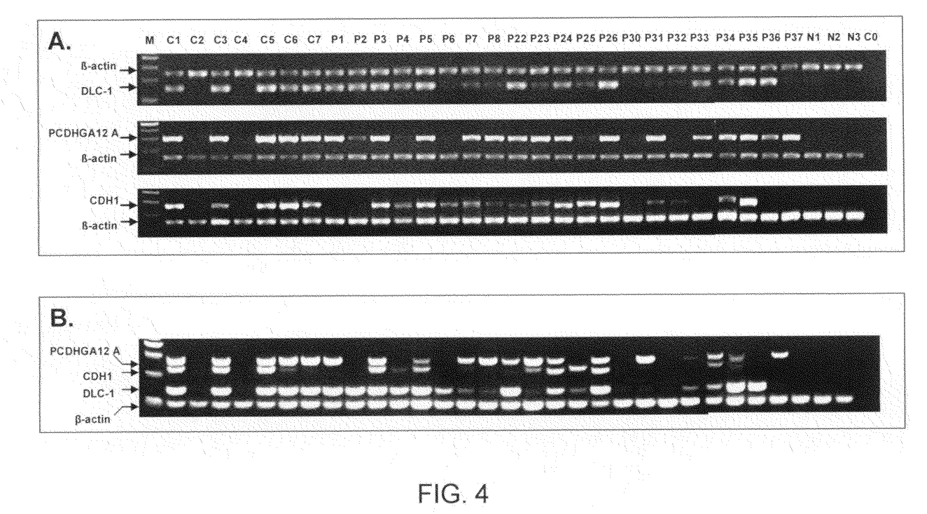

The invention also provides that the performing PCR amplification step may employ a multiplex PCR method with a combination of primer sets for all three selected genes (DLC-1, PCDHGA 12A, and CDH1) to prevent false negative results. Refer to FIGS. 4A and 4B, which are the PCR results for 21 B-ALL patient bone marrow samples. FIG. 4A shows three sets of testing results using the inventive method with the selected primers targeting each gene separately, while, FIG. 4B shows the multiplex PCR results using the inventive method with the selected primers targeting all three genes collectively. Optionally, the inventive method with multiplex PCR may be used as a cancer screening method. When adopting the multiplex PCR with the above selected three primer sets, the inventive method can detect leukemia cells in over 90% of B-ALL patient bone marrow biopsy specimens.

Furthermore, the performing a PCR amplification procedure may include a nested PCR procedure with a sequential set of pre-selected primers. According to one embodiment, the performing step includes the sub-steps of amplifying said digested DNA with a first pre-selected primer to result a first PCR product with long fragment, and then amplifying said first PCR product with a second pre-selected primer to result a second PCR product with short fragment. For example, when testing a DNA sample of a patient suspecting of ALL, the performing step may include first, amplifying the digested DNA with a pre-selected DLC-1 FF/AR primer (SEQ ID Nos 13 and 14) to result a first PCR product with 383 by fragment, and then, amplifying said 383 by fragment PCR product with a pre-selected DLC-1B primer (SEQ ID Nos 15 and 16) to result a second PCR product with 160 by fragment. With a nested PCR, the analytic sensitivity reaches 10⁻⁶ (1 cancer cell in 1 million normal cells). Optionally, the inventive method with nested PCR may be also used as a cancer screening method.

The invention demonstrates that the inventive method can achieve very high analytic sensitivity, which compassing two aspects, absolute sensitivity and relative sensitivity. Absolute sensitivity refers to the capability to detect minimal quality of methylated target DNA; relative sensitivity refers to the capability to detect the smallest fraction of methylated DNA in the presence of an excess of unmethylated DNA. The relative sensitivity represents a true sensitivity in tumor cell detection in patient clinical samples.

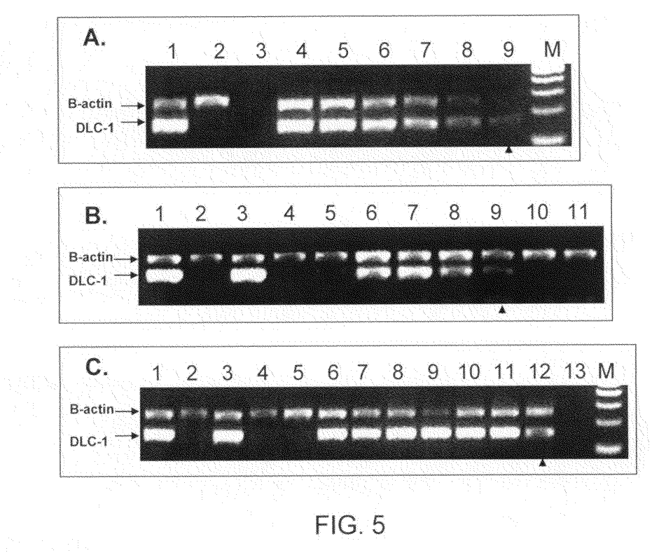

To determine the absolute analytic sensitivity of the inventive method in detecting certain ALL cancer cells, a 5× dilution series of digested genomic DNA of B-ALL cell line NALM-6 has been subjected to the inventive method. As shown in FIG. 5A, 0.03 ng tumor genomic DNA 5 cells) can be detected (lane 9), which is a significant improvement over the standard flow cytometry method that requires at least several thousand cells.

The relative sensitivity of the inventive method in detecting certain ALL cancer cells has also been examined, shown in FIG. 5B. Specifically, a 10× dilution series of NALM-6 DNA mixed with normal blood DNA to the total amount of 250 ng DNA has been employed as the starting samples. In FIG. 5B, a faint DLC-1 (160 bp) band can be seen at the sample with 0.25 ng of NALM-6 over 250 ng of normal DNA, which gives the sensitivity at about 10⁻³. The internal control β-actin band (257 bp) showed in all lanes with even density. This result suggested that the relative sensitivity of the inventive method using a single PCR is 0.1%, or 1 malignant allele in 1000 normal allele background.

To increase the relative sensitivity, a nested PCR has been performed. The 383 by long product (within regions 1 and 2 of DLC-1 CpG island, data not shown) as the first PCR product was amplified in the 2^{nd} PCR with results shown in FIG. 5C. The sensitivity has been dramatically increased to 10⁻⁶.

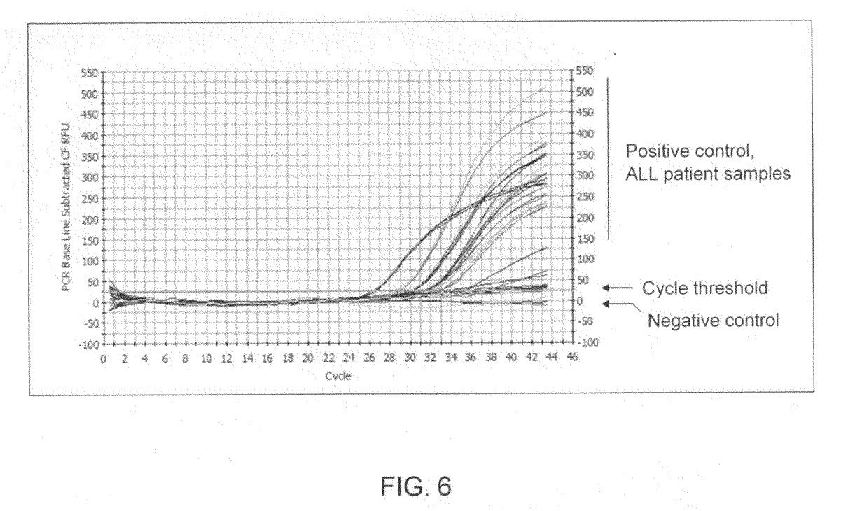

The PCR amplification step may further include the steps of carrying out a real-time PCR procedure to quantitatively determine the percentage of the malignant cells in a testing sample. Refer to FIG. 6, which is exemplary real-time PCR using the inventive method on four B-ALL patients' samples against the pre-selected positive and negative controls.

In the aforesaid determining step, the invention selected the known leukemia cell lines or Sss I CpG methyltransferase-treated normal human blood cell DNA as the positive control and the normal human blood or bone marrow cell DNA as the negative control. Both controls are processed following same extraction, digestion, and amplification protocols as the testing samples.

The invention further provides examples of employing the inventive method in detecting leukemia cells in ALL patient specimens.

Materials and Method:

Cell Lines:

All cell lines are human in origin. Precursor B-cell acute lymphoblastic leukemia (B-ALL) cell lines NALM-6, MN-60 and SD-1, and Precursor T-cell acute lymphoblastic leukemia (T-ALL) Jurkat cell line were purchased from DSMZ (Braunschweig, Germany). Non-Hodgkin lymphoma (NHL) cell lines Mec-1, Mec-2, and Wac-3 (chronic lymphocytic leukemia), RL (follicular lymphoma with t(14; 18) translocation), Granta-519 (mantle cell lymphoma with t(11; 14) translocation), Daudi, Raji (Burkett lymphoma), DB (diffuse large B-cell lymphoma) and RPMI 8226, KAS 6/1, NC1-H929, and U266B1 (plasma cell myeloma) were purchased from ATCC (Manassas, Va.). Control cell lines KG-1 and KG-1A (acute myeloid leukemia, ATCC) were also include in some experiments. All cell lines were maintained in RPMI 1640 medium supplemented with 10% FCS and 100 ug/ml of penicillin/streptomycin. The cells in exponential growth phase were collected and frozen at −80 C until DNA extraction.

Patients and Clinical Samples:

Bone marrow aspirates and blood samples were obtained from patients at diagnostic evaluation for suspected acute leukemia and at follow up visits after chemotherapy at the Ellis Fischel Cancer Center and Children's Hospital, University of Missouri Health Care (Columbia, Mo.), University of California at Irvin Medical Center (Irvin, Calif.) and University of Texas Southwestern Medical center (Dallas, Tex.) in compliance with local Institutional Review Board approvals. Aliquots of bone marrow mononuclear cells isolated with Ficoll-Hypaque gradient medium (Pharmacia Fine Chemicals) and whole blood in EDTA or citrate tube were stored in liquid nitrogen canister and at −80 C freezer, respectively, until use. Bone marrow differential count collected at the time of diagnosis and follow up visits are available. Flow cytometric immunophenotyping, cytogenetic analysis, and molecular clonality data in a subset of cases are also available.

General procedures for DNA isolation, multiple methylation sensitive enzyme digestion, PCR, nested PCR and Real-time PCR

The bone marrow mononuclear cells are thawed in 37° C. water bath and washed in 10 ml PBS by centrifugation at 100 g for 10 min. The frozen whole blood EDTA or citrate tubes are thawed in a 37° C. water bath. Genomic DNA is isolated using QIAamp DNA Blood mini kit (Qiagen, Valencia, Calif.) according to manufacture's instruction. Normal human male and female genomic DNAs from pooled human peripheral blood are purchased from Promega (Madison, Wis.).

To prepare positive control DNA, normal human genomic DNA is treated with M. Sss I CpG methyltransferase (New England Biolabs, Beverly, Mass.) that methylated all cytosine residues of CpG dinucleotides. The genomic DNAs or Sss I treated DNA (250 ng) is digested with 5U of methylation sensitive enzymes Aci I, Hap II, and HinP1 I (New England Biolabs, Ipswich, Mass.) in NEB Buffer 4 at 37° C. for 16 hours. Then 5U of BstU I (New England Biolabs, Ipswich, Mass.) is added, and the digestion is continued for additional 4 hours at 60° C. The enzymes are then inactivated at 65° C. for 20 min and the digested DNA was stored at −20° C. until use.

In a typical PCR, 40 ng of digested DNA, DCL-1 primers (0.5 uM) and β-actin primers (0.25 uM) were mixed with GoTaq Polymerase 2× green master mix (Promega, Madison, Wis.) in a final volume of 25 ul. The PCR reaction was carried out at PTC100 thermal cycler (MJ Research, Ramsey, Mich.) or equivalent instrument with the following program: denature at 95° C. for 30 s, anneal at 60° C. for 60 s, extension at 72° C. for 60 s for 35 cycles with 2 min at 95° C. for initial denaturation and 5 min at 72° C. for final extension. The selected target regions contain a total of 6-19 restriction sites to ensure a complete digestion. After digestion, the methylated target region in tumor cells will remain intact while the same unmethylated region in normal cells will be completely digested with multiple methylation sensitive enzymes. The protected methylated regions in tumor or leukemia cells will be amplified by PCR and the PCR product is visualized on 3% agarose gel stained with ethidium bromide or SYBR Green 1 fluorescence dye after electrophoresis. A region of β-actin gene free from enzyme cut-sites is amplified in each tube as an internal control for PCR reaction. Normal human genomic DNA with and without digestion, human genomic DNA with or without Sss I-treatment and B-ALL cell line NALM-6 genomic DNA are used as the digestion and methylation positive and negative controls, respectively.

In the nested PCR, the digested DNA was first amplified with DLC-1 FF/AR primers and yields a 383 by fragment product. The diluted first PCR product was used as a template and was amplified with DLC-1 BF/BR primer in 2^{nd} round PCR.

For the real-time PCR, the probes are labeled with FAM at 5′-end and BHQ1 at 3′-end. The sequences of the forward primer, reverse primer and the probe of DLC-1 are 5′-AGA ACA GGC ACG GAC TTG AC-3′, 5′-GAA AAC CCC GCT TTC TTT-3′ and 5′-FAM-GTT AGG ATC ATG GTG TCC GGC TTC TT-BHQ1-3′, respectively. The 40 ng of digested DNA, 500 nM of each primer, 250 nM of probe are mixed with a 2× master mix (ABsolute QPCR mix, ABgene, Surrey, UK) in a final volume of 25 ul. A two-step reaction protocol are carried out at iCycler BioRad instrument (Bio-Rad, Hercules, Calif.) with the program of 95° C. for 15 min (1 cycle for enzyme activation), 95° C. for 15 sec and 60° C. for 60 sec for 40 cycles. A region of 13-actin gene is amplified in a separate tube in each run for normalization. The percentage of leukemia cells in patient samples is calculated against the standard curve. The standard curve is constructed using a linear regression model over the linear range of six point dilution series.

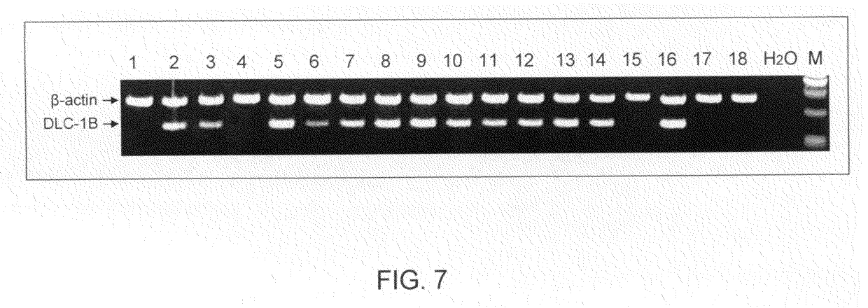

Referring to FIG. 7, the inventive method has been tested with 15 known leukemia/lymphoma cell lines, including B-ALL cell lines NALM-6, MN-60, and SD-1, CLL cell lines Mec-1, Mec-2 Wac-3, mantle cell lymphoma cell line Granta-519, follicular lymphoma cell line RL, diffuse large B-cell lymphoma DB, Burkett lymphoma cell lines Daudi, Raji, and plasma cell myeloma cell lines NC1-H929, RPMI 8226, U266B1, and KAS 6/1. As shown in FIG. 7, out of the 15 lymphoid malignant cells lines (line 2-16), 13 are detected by the inventive method, while only 2 (SD-1 lane 4) and U266B1 (lane 15) have failed the detection.

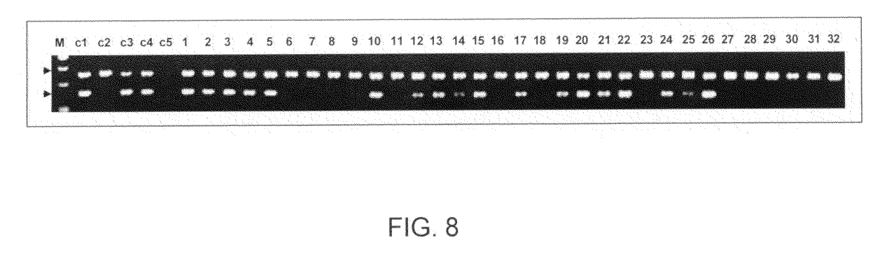

Refer to FIG. 8, which illustrates detection of DLC-1 methylation in B-ALL patients. In FIG. 8, a representative gel with 26 B-ALL diagnostic bone marrow aspirates was demonstrated. All DNAs were digested with 4 multiple methylation sensitive enzymes except lane c1 (non-digestion control). DLC-1 methylation was visualized on 3% agarose gel containing SYBR Green dye. Lane M: 100 by DNA ladder; Lane c1: normal human DNA non-digested; lane c2: normal human DNA digested (negative control); lane c3: methylase SssI-treated DNA (positive control); lane c4: B-ALL cell line NALM-6 DNA (positive control); lane c5: water (PCR negative control); lanes 1-26: B-ALL patient bone marrow DNA; lanes 27-32: normal bone marrow DNA. Upper arrow: β-actin bands, internal control; Lower arrow: DLC-1 methylation bands. DNA methylation of DLC-1 was detected in 18/26 (69%) in this B-ALL patient series (lanes 1-26), but not in normal human blood (lane c2) and 6 normal bone marrow samples (lanes 27-32).

While the invention has been described in connection with specific embodiments thereof, it will be understood that the inventive methodology is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth and as follows in scope of the appended claims.

### REFERENCES

1. Jones P A, Baylin S B: The epigenomics of cancer. Cell 128(4):683-92, 2007.
2. Laird P W: Cancer epigenetics. Hum Mol Genet, 15; 14 Spec No 1:R65-76, 2005.
3. Esteller, M, Profiling aberrant DMA methylation in hematologic neoplasms: a view from the tip of the iceberg, Clinical Immunology, 109: 80-88, 2003.
4. Lehmann U, Brakensiek K, Kreipe H, Role of epigenetic changes in hematologic malignancies, Ann Hematol, 83:137-152, 2004.
5. Galm O, Herman J G, Baylin S B: The fundamental role of epigenetics in hematopoietic malignancies, Blood Rev. 20(1):1-13, 2006.
6. Baylin S B, Ohm J E: Epigenetic gene silencing in cancer—a mechanism for early oncogenic pathway addiction? Nat Rev Cancer, 6(2):107-16, 2006.
7. Cotello J F et al: Aberrant CpG-island methylation has non-random and tumour-type-specific patterns, Nat Genet 24(2):132-8, 2000.
8. Roman-Gomez J, Castillejo J A, Jimenez A, Barrios M, Heiniger A, and Torres A, The role of DNA hypermethylation in the pathogenesis and prognosis of acute lymphoblastic leukemia, Leukemia & Lymphoma, 44:1855-1864,2003.
9. Yuan B Z, Durkin M E, Popescu N C: Promoter hypermethylation of DLC-1, a candidate tumor suppressor gene, in several common human cancers. Cancer Genet Cytogenet. 140(2):113-7, 2003.
10. Waha A, Giintner S, Huang T H, Yan P S, Arslan B, Pietsch T, Wiestler O D, Waha A: Epigenetic silencing of the protocadherin family member PCDH-gamma-A11 in astrocytomas. Neoplasia. 7(3):193-9, 2005.
11. Wang M X., Duff D., Shi H D, Taylor K H., Gruner B A., and Caldwell C W: Detection of Minimal Residual Disease in Precursor B Lymphoblastic Leukemia (B-ALL) Patients by a Novel Epigenetic DNA Methylation Biomarker, Blood, 106, Abstract 4524, 2005.
12. Wang, M X, Shi, H D, Zhao X H, Taylor K H, Duff D and Caldwell C W: Molecular Detection of Residual Leukemic Cells by a Novel DNA Methylation Biomarker (Abstract 1206), Modern Pathology 20 (supplement 2): 263A, 2007.
13. Taylor K H, Pena-Hernandez K E, Davis J W, Arthur G L, Duff D J, Shi H, Rahmatpanah F B, Sjahputera O, Caldwell C W: Large-scale CpG methylation analysis identifies novel candidate genes and reveals methylation hotspots in acute lymphoblastic leukemia. Cancer Res. 67(6):2617-25, 2007.
14. Shi H, Guo J, Duff D J, Rahmatpanah F, Chitima-Matsiga R, Al-Kuhlani M, Taylor K H, Sjahputera O, Andreski M, Wooldridge J E, Caldwell C W: Discovery of novel epigenetic markers in non-Hodgkin's lymphoma. Carcinogenesis 28(1):60-70, 2007
15. Ohm J E, McGarvey K M, Yu X, Cheng L, Schuebel K E, Cope L, Mohammad H P, Chen W, Daniel V C, Yu W, Berman D M, Jenuwein T, Pruitt K, Sharkis S J, Watkins D N, Herman J G, Baylin S B: A stem cell-like chromatin pattern may predispose tumor suppressor genes to DNA hypermethylation and heritable silencing. Nat Genet. 39(2):237-42, 2007.
16. Singer-Sam J, LeBon J M, Tanguay R L, Riggs A D: A quantitative HpaII-PCR assay to measure methylation of DNA from a small number of cells. Nucleic Acids Res. 11; 18(3):687, 1990.

1.-6. (canceled)

7. A method for the diagnosis, prognosis or detection of acute lymphoblastic leukemia (ALL), or of minimal residual disease (MRD) in acute lymphoblastic leukemia (ALL) patients, comprising:
contacting genomic DNA, obtained from a biological sample of a human subject and having at least one genomic DNA target sequence selected from the CpG island group consisting of DLC-1, PCDHGA 12, CDH1, and portions thereof, with a plurality of different methylation-sensitive restriction enzymes each having at least one CpG methylation-sensitive cleavage site within the at least one genomic DNA target sequence, wherein the at least one target sequence is either cleaved or not cleaved by each of said plurality of different methylation-sensitive restriction enzymes;
amplifying the contacted genomic DNA with at least one primer set defining an amplicon comprising the at least one target sequence, or the portion thereof, having the at least one CpG methylation-sensitive cleavage site for each of the plurality of different methylation-sensitive restriction enzymes to provide an amplificate; and
determining, based on a presence or absence of, or on a pattern or property of the at least one such amplificate relative to that of a normal control, a methylation state of at least one CpG dinucleotide sequence of the at least one target nucleic acid sequence, wherein a method for the diagnosis, prognosis or detection of acute lymphoblastic leukemia (ALL), or of minimal residual disease (MRD) in the human subject is afforded.

8. The method of claim 7, wherein the at least one target sequence comprises the DLC-1 gene CpG island or a portion thereof.

9. The method of claim 7, wherein said amplification comprises at least one of standard, multiplex, nested and real-time formats.

10. The method of claim 7, comprising amplification of a plurality of target sequences within the DLC-1 gene CpG island.

11. The method of claim 8, wherein the at least one target sequence additionally comprises at least one of the PCDHGA 12 gene CpG island, and portions thereof.

12. The method of claim 8, wherein the at least one target sequence additionally comprises at least one of the CDH1 gene CpG island, and portions thereof.

13. The method of claim 8, wherein the at least one target sequence additionally comprises the PCDHGA 12 and CDH1 CpG islands, or portions thereof.

14. The method of claim 7, wherein said methylation sensitive enzyme comprises at least one selected from the group consisting of Aci I, Hap II, HinP1 I, BstU I, Hha I, and Tai I.

15. The method of claim 7, wherein the at least one genomic DNA target sequence comprises at least 6 methylation-sensitive restriction sites.

16. The method of claim 7, wherein the at least one genomic DNA target sequence comprises at least four different methylation-sensitive restriction sites, and contacting comprises contacting the at least one genomic DNA target sequence with a respective four different methylation-sensitive restriction enzymes.

17. The method of claim 7, wherein the biological sample comprises at least one of blood and bone marrow.

18. The method of claim 7, comprising diagnosis or detection of acute lymphoblastic leukemia (ALL), or of minimal residual disease (MRD) in biofluids or tissue samples of either hematopoietic or solid tumors.

19. The method of claim 7, wherein the biological sample is from a post-chemotherapy subject.

20. The method of claim 7, wherein the relative sensitivity in detecting acute lymphoblastic leukemia (ALL), or minimal residual disease (MRD) is one malignant cell or allele in one million normal cells or alleles (10⁻⁶).

21. A method of determining CpG methylation status of genomic DNA, comprising: contacting genomic DNA, obtained from a biological sample of a subject and having at least one genomic DNA target sequence, with a plurality of different methylation-sensitive restriction enzymes each having at least one CpG methylation-sensitive cleavage site within the at least one genomic DNA target sequence, wherein the at least one target sequence is either cleaved or not cleaved by each of said plurality of different methylation-sensitive restriction enzymes; amplifying the contacted genomic DNA with at least one primer set defining an amplicon comprising the at least one target sequence, or a portion thereof, having the at least one CpG methylation-sensitive cleavage site for each of the plurality of different methylation-sensitive restriction enzymes to provide an amplificate; and determining, based on a presence or absence of, or on a pattern or property of the at least one such amplificate relative to that of positive and negative controls, a methylation state of at least one CpG dinucleotide sequence of the at least one target nucleic acid sequence, wherein a method for determining CpG methylation status of genomic DNA is afforded.

22. The method of claim 21, wherein said methylation sensitive enzyme comprises at least one selected from the group consisting of Aci I, Hap II, HinP1 I, BstU I, Hha I, and Tai I.

23. The method of claim 21, wherein the at least one genomic DNA target sequence comprises at least 6 methylation-sensitive restriction sites.

24. The method of claim 21, wherein the at least one genomic DNA target sequence comprises at least four different methylation-sensitive restriction sites, and contacting comprises contacting the at least one genomic DNA target sequence with a respective four different methylation-sensitive restriction enzymes.

25. The method of claim 21, wherein said amplification comprises at least one of standard, multiplex, nested and real-time formats.

26. The method of claim 21, wherein the relative sensitivity in detecting CG methylation status is one methylated allele in one million non-methylated alleles (10⁻⁶).


SEQUENCE LISTING (17 sequences)

SEQ ID NO: 1 - DNA, 20 bp, Artificial Sequence
  targeting region 1 - DLC1-WF
gaaagtgaaccagggcttcc

SEQ ID NO: 2 - DNA, 24 bp, Artificial Sequence
  targeting region1 - DLC1-BR
tgcttgatgtgcagaaagaagccg

SEQ ID NO: 3 - DNA, 24 bp, Artificial Sequence
  targeting region 2 - DLC1-AF
tgttaggatcatggtgtccggctt

SEQ ID NO: 4 - DNA, 24 bp, Artificial Sequence
  targeting region 2- DLC1-AR
agcgctccctcgtttcgatcttta

SEQ ID NO: 5 - DNA, 20 bp, Artificial Sequence
  targeting region 3 - DLC1-EF
cagaaagaaagcggggtttt

SEQ ID NO: 6 - DNA, 18 bp, Artificial Sequence
  targeting region 3 - DLC1-WR
taaggcctgcgacccaga

SEQ ID NO: 7 - DNA, 24 bp, Artificial Sequence
  targeting region 4 - PCDHGA12-AF
actcacttctccctcatcgtgcaa

SEQ ID NO: 8 - DNA, 24 bp, Artificial Sequence
  targeting region 4 - PCDHGA12-AR
acctcacttccgcattgactcctt

SEQ ID NO: 9 - DNA, 24 bp, Artificial Sequence
  targeting CDH1 (forward primer)
tgagcttgcggaagtcagttcaga

SEQ ID NO: 10 - DNA, 24 bp, Artificial Sequence
  targeting CDH1 (reverse primer)
ttcttggaagaagggaagcggtga

SEQ ID NO: 11 - DNA, 24 bp, Artificial Sequence
  targeting beta-actin (forward primer)
ggccgaggactttgattgcacatt

SEQ ID NO: 12 - DNA, 24 bp, Artificial Sequence
  targeting beta-actin (reverse primer)
gggcacgaaggctcatcattcaaa

SEQ ID NO: 13 - DNA, 25 bp, Artificial Sequence
  nested PCR for DLC1 (forward primer)
aaatccggagactctgcagaaagcg

SEQ ID NO: 14 - DNA, 24 bp, Artificial Sequence
  nested PCR DLC1-BF (forward primer)
taaagagcacagaacaggcaccga

SEQ ID NO: 15 - DNA, 20 bp, Artificial Sequence
  Real-time PCR primer for DLC1 (forward primer)
agaacaggcacggacttgac

SEQ ID NO: 16 - DNA, 18 bp, Artificial Sequence
  Real-time PCR for DLC1 (reverse primer)
gaaaaccccgctttcttt

SEQ ID NO: 17 - DNA, 26 bp, Artificial Sequence
  Real-time PCR for DLC1 (probe primer) 5′-FAM and 3′-BHQ1
gttaggatcatggtgtccggcttctt